(19) United States
(12) Patent Application Publication
(10) Pub. No.: US 2019/0125313 A1
(43) Pub. Date: May 2, 2019

(54) WIRELESS CHARGER FOR ULTRASOUND IMAGING DEVICE

(71) Applicant: Butterfly Network, Inc., Guilford, CT (US)

(72) Inventors: Dominick Mastri, Bridgeport, CT (US); Matthew de Jonge, New York, NY (US); Stephen Christopher, Philadelphia, PA (US); Paul Maxted, Guilford, CT (US); Matthew R. Hageman, Hoffman Estates, IL (US)

(73) Assignee: Butterfly Network, Inc., Guilford, CT (US)

(21) Appl. No.: 16/171,655

(22) Filed: Oct. 26, 2018

Related U.S. Application Data
(60) Provisional application No. 62/578,238, filed on Oct. 27, 2017.

(51) Int. Cl. A61B 8/00; H02J 50/90; H02J 50/10; H02J 7/02

(52) U.S. Cl. CPC A61B 8/56; A61B 8/4472; H02J 7/025; H02J 50/90; H02J 50/10; A61B 8/4433

(57) ABSTRACT

Described herein are chargers for wirelessly charging an ultrasound imaging device. The charger may include a base member, a transmitter coil coupled to the base member, and a cradle extending from the top surface of the base member and including a surface configured to receive the ultrasound imaging device, where the first surface is disposed a vertical distance above the top surface of the base member. The cradle may be configured such that no portion of the ultrasound imaging device contacts the top surface of the base member when the cradle holds the ultrasound imaging device. The cradle may be configured to hold the ultrasound imaging device so as to substantially maintain alignment of a receiver coil of the ultrasound imaging device with the transmitter coil.

### CROSS-REFERENCE TO RELATED APPLICATIONS

This application claims the benefit under 35 U.S.C. § 119(e) of U.S. Provisional Patent Application Ser. No. 62/578,238, filed Oct. 27, 2017 under Attorney Docket No. B1348.70059US00 and entitled “WIRELESS CHARGER FOR ULTRASOUND DEVICE,” which is hereby incorporated herein by reference in its entirety.

### FIELD

Generally, the aspects of the technology described herein relate to ultrasound systems. Some aspects relate to a charger for wirelessly charging an ultrasound imaging device.

### BACKGROUND

Ultrasound devices may be used to perform diagnostic imaging and/or treatment, using sound waves with frequencies that are higher with respect to those audible to humans.

Ultrasound imaging may be used to see internal soft tissue body structures. When pulses of ultrasound are transmitted into tissue (e.g., by using an ultrasound imaging device), sound waves are reflected off the tissue, with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, an apparatus for wirelessly charging an ultrasound imaging device includes a base member having a top surface, a transmitter coil disposed within the base member, and a cradle extending from the top surface of the base member and configured to hold the ultrasound imaging device so as to substantially maintain alignment of a receiver coil of the ultrasound imaging device with the transmitter coil. In some embodiments, the cradle is configured to hold the ultrasound imaging device such that a center portion of the receiver coil is positioned substantially directly above a center portion of the transmitter coil.

In some embodiments, the cradle further includes a first cradle member having a first surface configured to conform to a first portion of the ultrasound imaging device and a second cradle member including a second surface configured to conform to a second portion the ultrasound imaging device, where the first surface of the first cradle member and the second surface of the second cradle member are configured to simultaneously conform to the first portion and the second portion of the ultrasound imaging device, respectively, so as to position the center portion of the receiver coil is substantially directly above the center portion of the transmitter coil. In some embodiments, the first surface of the first cradle member is concave and faces a same direction as the top surface of the base member and the second surface of the second cradle member is concave and faces the same direction as the top surface of the base member.

In some embodiments, a first radius of curvature of the first surface of the first cradle member is greater than a second radius of curvature of the second surface of the second cradle member. In some embodiments, a width of the first surface is between approximately 1/16-2 inches. In some embodiments, a distance between the first cradle member and the second cradle member along a longitudinal axis of the base member is between approximately 2-5 inches.

According to another aspect, a system for wirelessly charging an ultrasound imaging device includes an ultrasound imaging device and a charger. The charger includes a base member having a top surface, a transmitter coil disposed within the base member, and a cradle extending from the top surface of the base member and configured to hold the ultrasound imaging device so as to substantially maintain alignment of a receiver coil of the ultrasound imaging device with the transmitter coil. In some embodiments, the cradle is configured to hold the ultrasound imaging device such that a center portion of the receiver coil is positioned substantially directly above a center portion of the transmitter coil.

In some embodiments, the cradle further includes a first cradle member having a first surface configured to conform to a first portion of the ultrasound imaging device and a second cradle member including a second surface configured to conform to a second portion the ultrasound imaging device, where the first surface of the first cradle member and the second surface of the second cradle member are configured to simultaneously conform to the first portion and the second portion of the ultrasound imaging device, respectively, so as to position the center portion of the receiver coil is substantially directly above the center portion of the transmitter coil.

In some embodiments, a first distance along a longitudinal axis of the ultrasound imaging device from the first portion of the ultrasound imaging device to the center portion of the receiver coil is substantially equivalent to a second distance along a longitudinal axis of the base member from the first cradle member to the center portion of the transmitter coil. In some embodiments, a first distance along a longitudinal axis of the ultrasound imaging device from the first portion of the ultrasound imaging device to the second portion of the ultrasound imaging device is substantially equivalent to a second distance along a longitudinal axis of the base member from the first cradle member to the second cradle member. In some embodiments, a first radius of curvature of the ultrasound imaging device at the first portion of the ultrasound imaging device is substantially equivalent to a second radius of curvature of the first surface of the first cradle member, and a third radius of curvature of the ultrasound imaging device at the second portion of the ultrasound imaging device is substantially equivalent to a fourth radius of curvature of the second surface of the second cradle member.

In some embodiments, the first surface of the first cradle member is concave and faces a same direction as the top surface of the base member and the second surface of the second cradle member is concave and faces the same direction as the top surface of the base member. In some embodiments, a first radius of curvature of the first surface of the first cradle member is greater than a second radius of curvature of the second surface of the second cradle member. In some embodiments, a width of the first surface is between approximately 1/16-2 inches. In some embodiments, a distance between the first cradle member and the second cradle member along a longitudinal axis of the base member is between approximately 2-5 inches.

According to another aspect, an apparatus for wirelessly charging an ultrasound imaging device includes a base member having a top surface, a transmitter coil disposed within the base member, and a cradle extending from the top surface of the base member and configured to hold the ultrasound imaging device such that no portion of the ultrasound imaging device contacts the top surface of the base member.

In some embodiments, the cradle includes a first cradle member having a first surface that is concave and configured to receive a first portion of the ultrasound imaging device. In some embodiments, the first surface includes a first portion that is substantially closest to the top surface of the base member and is disposed a first vertical distance above the top surface of the base member. In some embodiments, the cradle further includes a second cradle member spaced a distance along the longitudinal axis of the base member from the first cradle member and having a second surface that is concave and configured to simultaneously receive a second portion of the ultrasound imaging device when the first surface of the first cradle member receives the first portion of the ultrasound imaging device. In some embodiments, the second surface includes a second portion that is substantially closest to the top surface of the base member and is disposed a second vertical distance above the top surface of the base member.

In some embodiments, a first radius of curvature of the first surface of the first cradle member is greater than a second radius of curvature of the second surface of the second cradle member. In some embodiments, the second vertical distance is greater than the first vertical distance. In some embodiments, a distance between the first cradle member and the second cradle member along a longitudinal axis of the base member is between approximately 2-5 inches. In some embodiments, a width of the first surface is between approximately 1/16-2 inches.

According to another aspect, a system for wirelessly charging an ultrasound imaging device includes an ultrasound imaging device and a charger. The charger includes a base member having a top surface, a transmitter coil disposed within the base member, and a cradle extending from the top surface of the base member and configured to hold the ultrasound imaging device such that no portion of the ultrasound imaging device contacts the top surface of the base member.

In some embodiments, the cradle includes a first cradle member having a first surface that is concave and configured to receive a first portion of the ultrasound imaging device. In some embodiments, the first surface includes a first portion that is substantially closest to the top surface of the base member and is disposed a first vertical distance above the top surface of the base member. In some embodiments, the cradle further includes a second cradle member spaced a distance along the longitudinal axis of the base member from the first cradle member and having a second surface that is concave and configured to simultaneously receive a second portion of the ultrasound imaging device when the first surface of the first cradle member receives the first portion of the ultrasound imaging device. In some embodiments, the second surface includes a second portion that is substantially closest to the top surface of the base member and is disposed a second vertical distance above the top surface of the base member.

In some embodiments, a first radius of curvature of the first surface of the first cradle member is greater than a second radius of curvature of the second surface of the second cradle member. In some embodiments, the second vertical distance is greater than the first vertical distance. In some embodiments, a distance between the first cradle member and the second cradle member along a longitudinal axis of the base member is between approximately 2-5 inches. In some embodiments, a width of the first surface is between approximately 1/16-2 inches.

In some embodiments, a vertical distance from the top surface of the base member to a portion of the ultrasound imaging device that is substantially closest to the top surface of the base member when the cradle holds the ultrasound imaging device is between approximately 0.001-0.02 inches. In some embodiments, a vertical distance from a center portion of the transmitter coil to a center portion of a receiver coil in the ultrasound imaging device when the cradle holds the ultrasound imaging device is between approximately 0.03-0.3 inches.

According to another aspect, an apparatus for wirelessly charging an ultrasound imaging device includes a base member having a top surface, a transmitter coil disposed within the base member, and a cradle extending from the top surface of the base member, the cradle having a first surface configured to receive the ultrasound imaging device, where the first surface includes a first portion that is substantially closest to the top surface of the base member and is disposed a first vertical distance above the top surface of the base member.

In some embodiments, the cradle includes a first cradle member, the first cradle member includes the first surface, and the first surface is concave and faces a same direction as the top surface of the base member. In some embodiments, the cradle further includes a second cradle member, the second cradle member includes a second surface configured to simultaneously receive the ultrasound imaging device when the first surface of the first cradle member receives the ultrasound imaging device, the second surface is concave and faces the same direction as the top surface of the base member, and the second surface includes a second portion that is substantially closest to the top surface of the base member and is disposed a second vertical distance above the top surface of the base member.

In some embodiments, a first radius of curvature of the first surface of the first cradle member is greater than a second radius of curvature of the second surface of the second cradle member. In some embodiments, the second vertical distance is greater than the first vertical distance. In some embodiments, a distance between the first cradle member and the second cradle member along a longitudinal axis of the base member is between approximately 2-5 inches. In some embodiments, a width of the first surface is between approximately 1/16-2 inches.

According to another aspect, a system for wirelessly charging an ultrasound imaging device includes an ultrasound imaging device and a charger. The charger includes a base member having a top surface, a transmitter coil disposed within the base member, and a cradle extending from the top surface of the base member, the cradle having a first surface configured to receive the ultrasound imaging device, where the first surface includes a first portion that is substantially closest to the top surface of the base member and is disposed a first vertical distance above the top surface of the base member.

In some embodiments, the cradle includes a first cradle member, the first cradle member includes the first surface, and the first surface is concave and faces a same direction as the top surface of the base member. In some embodiments, the cradle further includes a second cradle member, the second cradle member includes a second surface configured to simultaneously receive the ultrasound imaging device when the first surface of the first cradle member receives the ultrasound imaging device, the second surface is concave and faces the same direction as the top surface of the base member, and the second surface includes a second portion that is substantially closest to the top surface of the base member and is disposed a second vertical distance above the top surface of the base member.

In some embodiments, a first radius of curvature of the first surface of the first cradle member is greater than a second radius of curvature of the second surface of the second cradle member. In some embodiments, the second vertical distance is greater than the first vertical distance. In some embodiments, a distance between the first cradle member and the second cradle member along a longitudinal axis of the base member is between approximately 2-5 inches. In some embodiments, a width of the first surface is between approximately 1/16-2 inches.

In some embodiments, the top surface of the base member is configured to not contact a portion of the ultrasound imaging device that is substantially closest to the top surface of the base member when the cradle receives the ultrasound imaging device. In some embodiments, a vertical distance from the top surface of the base member to a portion of the ultrasound imaging device that is substantially closest to the top surface of the base member when the cradle receives the ultrasound imaging device is between approximately 0.001-0.02 inches. In some embodiments, a vertical distance from a center portion of the transmitter coil to a center portion of a receiver coil in the ultrasound imaging device when the cradle receives the ultrasound imaging device is between approximately 0.03-0.3 inches.

According to another aspect, a charger for wirelessly charging an ultrasound imaging device includes: a) a base member that includes a cavity having an internal top surface, b) a compressible element disposed within the cavity, and c) a transmitter coil disposed within the cavity of the base member such that the transmitter coil is disposed between the internal top surface of the cavity and the compressible element, where the compressible element is configured to press the transmitter coil against the internal top surface. In some embodiments, the compressible element includes a rubber material. In some embodiments, a thickness of the compressible material is between approximately 0.05-1 inches.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 shows an example charger for wirelessly charging an ultrasound imaging device in accordance with certain embodiments described herein;

FIG. 2 shows an example side view of the charger of FIG. 1 holding the ultrasound imaging device of FIG. 1 in accordance with certain embodiments described herein;

FIGS. 3A-3B show an example top view of the charger of FIG. 1 holding the ultrasound imaging device of FIG. 1 in accordance with certain embodiments described herein;

FIG. 4 shows an example cross-section of the charger of FIG. 1 holding the ultrasound imaging device of FIG. 1 along the axis A-A of FIG. 3A in accordance with certain embodiments described herein;

FIG. 5 shows an example cross-section of the charger of FIG. 1 holding the ultrasound imaging device of FIG. 1 along the axis B-B of FIG. 3A in accordance with certain embodiments described herein;

FIG. 6 shows an example exploded view of the charger of FIG. 1 in accordance with certain embodiments described herein;

FIG. 7 shows an example cross-section of the charger of FIG. 1 holding the ultrasound imaging device of FIG. 1 along the axis C-C of FIG. 3B;

FIG. 8 shows an example cross-section of the charger of FIG. 1 holding the ultrasound imaging device of FIG. 1 along the axis D-D of FIG. 3A;

FIG. 9 shows another example charger in accordance with certain embodiments described herein;

FIG. 10 shows another example charger in accordance with certain embodiments described herein;

FIG. 11 shows another example charger in accordance with certain embodiments described herein;

FIG. 12 shows another example charger in accordance with certain embodiments described herein; and

FIG. 13 shows another example charger in accordance with certain embodiments described herein.

### DETAILED DESCRIPTION

Conventional ultrasound systems are large, complex, and expensive systems that are typically only purchased by large medical facilities with significant financial resources. Recently, cheaper, portable, and less complex ultrasound imaging devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application) and published as U.S. Patent Publication No. 2017/0360397 A1, which is incorporated by reference herein in its entirety. The reduced cost and increased portability of these new ultrasound devices may make them significantly more accessible to the general public than conventional ultrasound devices.

The inventors have recognized that portable ultrasound image devices may benefit from the convenience of wireless charging systems. Conventional wireless charging systems typically include a pad on which a device is placed. A coil in the pad inductively transmits energy to a coil in the device in order to charge the device's battery.

The inventors have further recognized that it may be helpful to configure a charger for an ultrasound imaging device so that the ultrasound imaging device does not contact the base member of the charger, which includes the transmitter coil. Instead, the inventors have recognized that it may helpful to configure the charger such that the ultrasound imaging device is raised above the top surface of the base member by a distance. This may help to increase air flow between the ultrasound imaging device and the top surface of the base member of the charger, increase the rate and degree of cooling of the ultrasound imaging device, and reduce the temperature of the ultrasound imaging device during and after wireless charging.

The inventors have further recognized that it may be helpful to configure a charger in ways that increase energy transfer efficiency between the transmitter coil in the base member of the charger and the receiver coil in the ultrasound imaging device. The inventors have recognized that various dimensions of the charger may be selected such that there may only be one position of the ultrasound imaging device relative to the charger in which the charger conforms to the ultrasound imaging device. Furthermore, the charger may be configured such that in the configuration in which the charger conforms to the ultrasound imaging device, the center portion of the receiver coil will be positioned substantially directly above the center portion of the transmitter coil. Positioning the center portion of the receiver coil directly above the center portion of the transmitter coil may help to decrease the distance from the receiver coil to the transmitter coil and thereby increase energy transfer efficiency between the transmitter coil and the receiver coil of the ultrasound imaging device.

Additionally, the inventors have recognized that it may be possible to configure the charger such that a compressible element presses the transmitter coil of the charger against an internal top surface of the base member of the charger, where the external top surface of the base member of the charger faces the ultrasound imaging device when the cradle holds the ultrasound imaging device. This may help to position the transmitter coil of the charger as close as possible to the receiver coil of the ultrasound imaging device, which may help to increase energy transfer efficiency.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

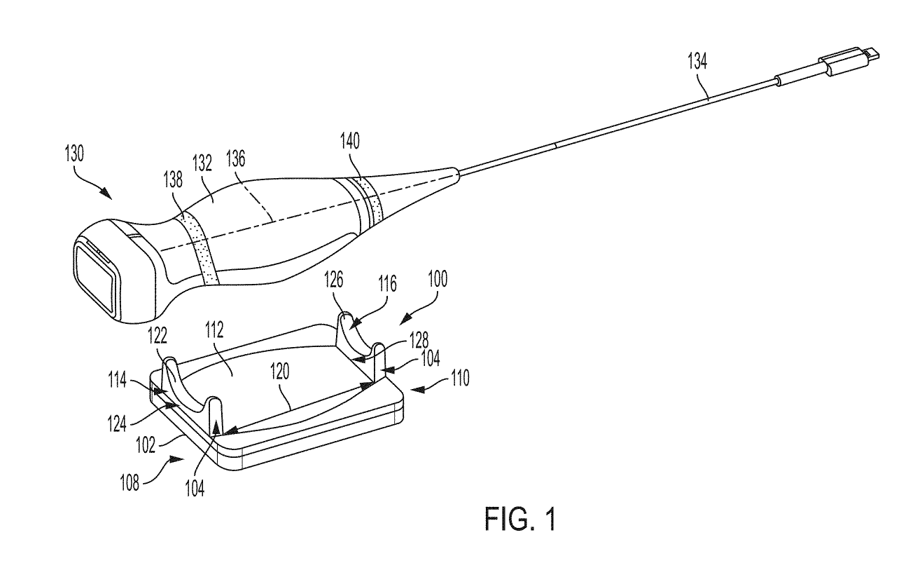

FIG. 1 shows an example charger 100 for wirelessly charging an ultrasound imaging device 130 in accordance with certain embodiments described herein. In FIG. 1, the charger 100 and the ultrasound imaging device 130 are shown prior to placement of the ultrasound imaging device 130 in the charger 100. The charger includes a base member 102 and a cradle 104. The base member 102 includes a first end 108, a second end 110, and a top surface 112. The cradle 104 extends from the top surface 112 of the base member 102 and includes a first cradle member 114 at the first end 108 and a second cradle member 116 at the second end 110. The first cradle member 114 and the second cradle member 116 are separated from each other by a distance 120 along the longitudinal axis of the charger 100. The distance 120 may be, for example, between approximately 2-5 inches (e.g., 2, 2.5, 3, 3.125, 3.25, 3.5, 3.75, 4, 4.5, 5 inches, or any suitable distance). The first cradle member 114 includes a first surface 122 that is concave and faces the same direction as the top surface 112 of the base member 102. The portion of the first surface 122 that is substantially closest to the top surface 112 of the base member 102 is disposed a vertical distance 124 above the top surface 112 of the base member 102. A particular portion of a first element may be considered substantially closest to a portion of a second element when no other portion of the first element is closer to the portion of the second element by a certain percentage of the distance between the particular portion of the first element and the portion of the second element. For example, the percentage may be 1%, 2%, 5%, 10%, or 20%, although other percentages are possible. The second cradle member 116 includes a second surface 126 that is concave and faces the same direction as the top surface 112 of the base member 102. The portion of the second surface 126 that is substantially closest to the top surface 112 of the base member 102 is disposed a vertical distance 128 above the top surface 112 of the base member 102.

FIG. 1 further shows the ultrasound imaging device 130, which includes a device body 132 and a cable 134. The device body 132 has a longitudinal axis 136. The device body 132 is shaped such that certain cross-sections of the device body 132 orthogonal to the longitudinal axis 136 have varying diameters.

The first cradle member 114 and the second cradle member 116 are configured to simultaneously receive and hold in place the ultrasound imaging device 130 when a first portion 138 of the ultrasound imaging device 130 rests on the first surface 122 of the first cradle member 114 and a second portion 140 of the ultrasound imaging device 130 rests on the second surface 126 of the second cradle member 116. The radius of curvature of the first surface 122, the radius of curvature of the second surface 126, the distance 120 between the first cradle member 114 and the second cradle member 116, the vertical distance 124, and/or the vertical distance 128 may be selected such that when the first portion 138 of the ultrasound imaging device 130 rests on the first surface 122 of the first cradle member 114 and the second portion 140 of the ultrasound imaging device 130 rests on the second surface 126 of the second cradle member 116, the first surface 122 of the first cradle member 114 conforms to the first portion 138 of the ultrasound imaging device 130 and the second surface 126 of the second cradle member 116 conforms to the second portion 140 of the ultrasound imaging device 130. For example, the distance along the longitudinal axis 136 of the ultrasound imaging device 130 from the first portion 138 of the ultrasound imaging device 130 to the second portion 140 of the ultrasound imaging device 130 may be substantially equivalent to the distance 120 from the first cradle member 114 to the second cradle member 116. Two values A and B may be considered substantially equivalent to each other when the absolute difference between A and B is less than a certain percentage of B, and/or when the absolute difference between A and B is less than a certain percentage of A. For example, the percentage may be 1%, 2%, 5%, 10%, 15%, or 20%, although other percentages are possible. As another example, the radius of curvature of the first portion 138 of the ultrasound imaging device 130 may be substantially equivalent to the radius of curvature of the first surface 122 of the first cradle member 114 and the radius of curvature of the second portion 140 of the ultrasound imaging device 130 may be substantially equivalent to the radius of curvature of the second surface 126 of the second cradle member 116. In some embodiments, the radius of curvature of the first cradle member 114, the radius of curvature of the second cradle member 116, the distance 120 between the first cradle member 114 and the second cradle member 116, the vertical distance 124, and/or the vertical distance 128 may be selected such that there may only be one position of the ultrasound imaging device 130 relative to the charger 100 in which the first surface 122 and the second surface 126 simultaneously conform to the ultrasound imaging device 130. In other words, the only portions of the ultrasound imaging device 130 to which the first surface 122 and the second surface 126 can simultaneously conform may be the first portion 138 and the second portion 140 of the ultrasound imaging device 130, respectively. As referred to herein, a first element “conforming” to a second element should be understood to mean that the second element contacts substantially all of the first element. Contacting substantially all of an element may mean contacting at least 99%, 98%, 95%, 90%, 85%, or 80% of the element, although other portions are possible. For simplicity, references to a charger or a cradle conforming to an ultrasound imaging device should be understood to mean that the surface(s) of the charger's cradle simultaneously conform to the ultrasound imaging device.

In some embodiments, the radius of curvature of the first surface 122 of the first cradle member 114 may be greater than the radius of curvature of the second surface 126 of the second cradle member 116. In some embodiments, the radius of curvature of the first surface 122 of the first cradle member 114 may be less than the radius of curvature of the second surface 126 of the second cradle member 116. In some embodiments, the radius of curvature of the first surface 122 of the first cradle member 114 may be equal to the radius of curvature of the second surface 126 of the second cradle member 116. In some embodiments, the vertical distance 128 may be greater than the vertical distance 124. In some embodiments, the vertical distance 128 may be less than the vertical distance 124. In some embodiments, the vertical distance 128 may be equal to the vertical distance 124. The width of the first surface 122 and the second surface 126 may be, for example, between approximately 1/16-2 inches (e.g., 1/16, ⅛/¼, ½, 1, 1.5, 2 inches, or any suitable width). In some embodiments, the first surface 122 may slope downwards, towards the top surface 112 of the base member 102, from the first end 108 of the base member 102 to the second end 110 of the base member 102. In some embodiments, the second surface 126 may slope downwards, towards the top surface 112 of the base member 102, from the second end 110 of the base member 102 to the first end 108 of the base member 102.

The vertical distance 124 and/or the vertical distance 128 may be selected such that, when the first cradle member 114 and the second cradle member 116 hold the ultrasound imaging device 130, no portion of the ultrasound imaging device 130 contacts the top surface 112 of the base member 102. This may be helpful because the ultrasound imaging device 130 and/or the charger 100 may generate heat when the charger 100 holds the ultrasound imaging device 130 during wireless charging of the ultrasound imaging device 130 by the charger 100, and ensuring that no portion of the ultrasound imaging device 130 contacts the top surface 112 of the base member 102 when the charger 100 holds the ultrasound imaging device 130 may help to increase air flow between the ultrasound imaging device 130 and the top surface 112 of the base member 102, increase the rate and degree of cooling of the ultrasound imaging device 130, and reduce the temperature of the ultrasound imaging device 130 during and after wireless charging.

In some embodiments the cradle 104 may include only one cradle member and surface for holding the ultrasound imaging device, or more than two cradle members and surfaces for holding the ultrasound imaging device (e.g., three, four, five, etc.). More generally, constructions other than a cradle may be used in some embodiments. According to an aspect of the present application, an ultrasound imaging device holder comprising one or more raised members may be used. A cradle is a non-limiting example.

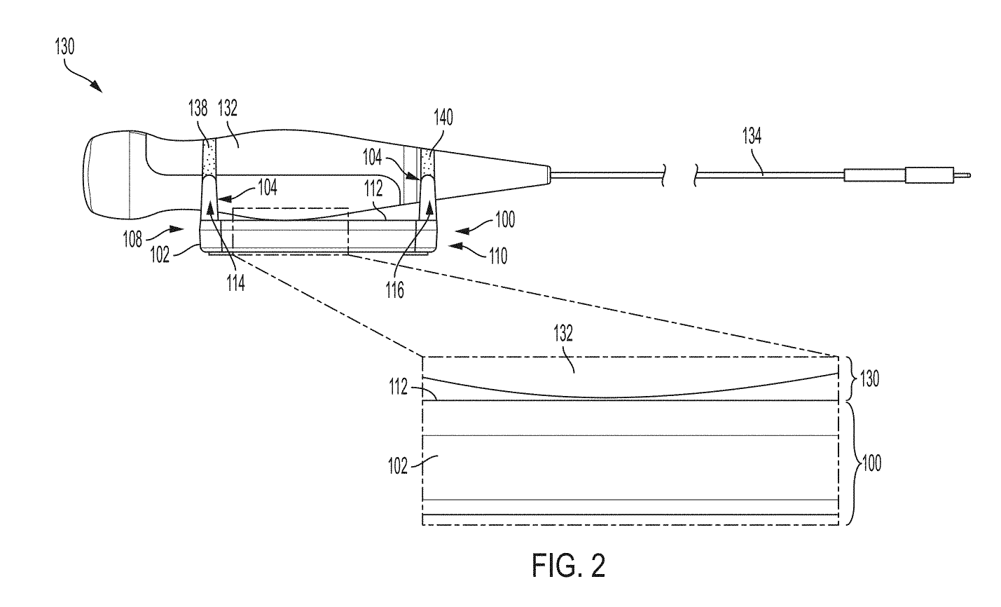

FIG. 2 shows an example side view of the charger 100 holding the ultrasound imaging device 130 in accordance with certain embodiments described herein, as well as a magnification of certain portions of the charger 100 and the ultrasound imaging device 130. As can be seen, the ultrasound imaging device 130 does not contact the top surface 112 of the base member 102. In some embodiments, a distance between the top surface 112 of the base member 102 and a portion of the ultrasound imaging device 130 that is substantially closest to the top surface 112 of the base member 102 when the cradle 104 holds the ultrasound imaging device 130 is 0.001-0.02 inches (e.g., 0.001 inches, 0.002 inches, 0.003 inches, 0.004 inches, 0.005 inches, 0.075 inches, 0.01 inches, 0.15 inches, 0.02 inches, or any suitable distance).

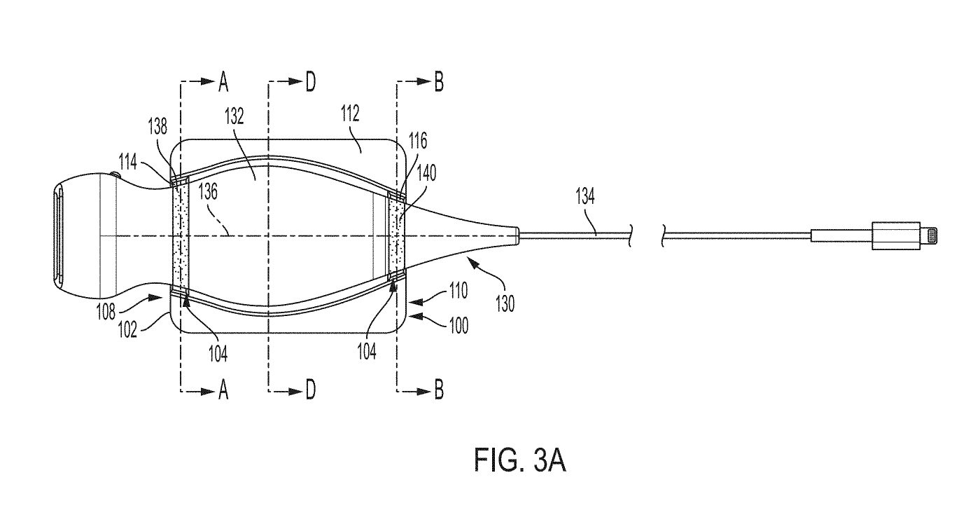

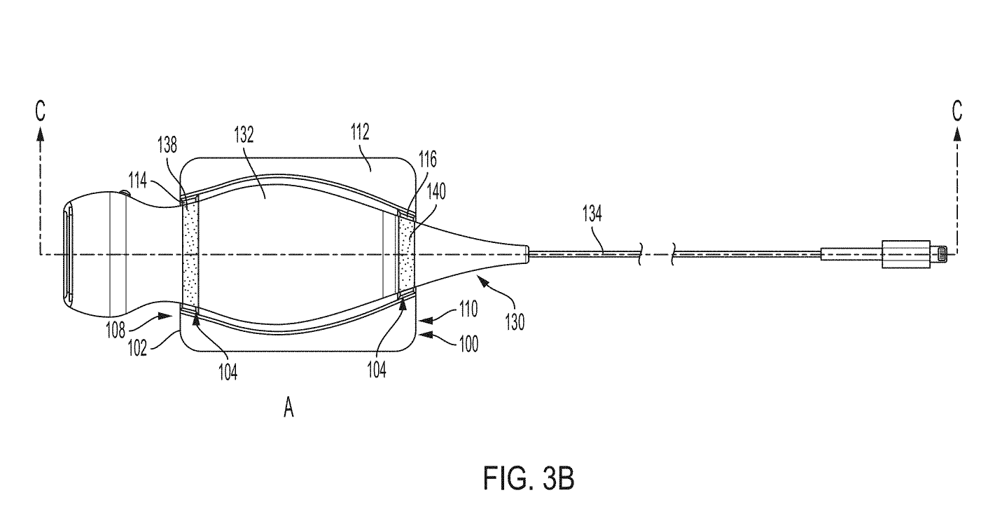

FIGS. 3A-3B show an example top view of the charger 100 holding the ultrasound imaging device 130 in accordance with certain embodiments described herein. FIGS. 3A-3B further shows axes A-A, B-B, C-C, and D-D along which cross-sections of the charger 100 and the ultrasound imaging device 130, viewed in the direction of the arrows, are shown in following figures.

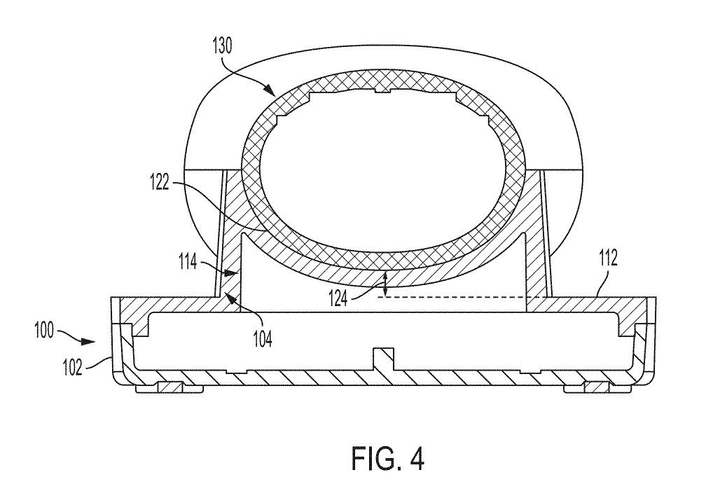

FIG. 4 shows an example cross-section of the charger 100 holding the ultrasound imaging device 130 along the axis A-A of FIG. 3A in accordance with certain embodiments described herein. For simplicity, certain internal components and structures of the charger 100 and the ultrasound imaging device 130 have been omitted. As can be seen, the first surface 122 of the first cradle member 114 conforms to the ultrasound imaging device 130.

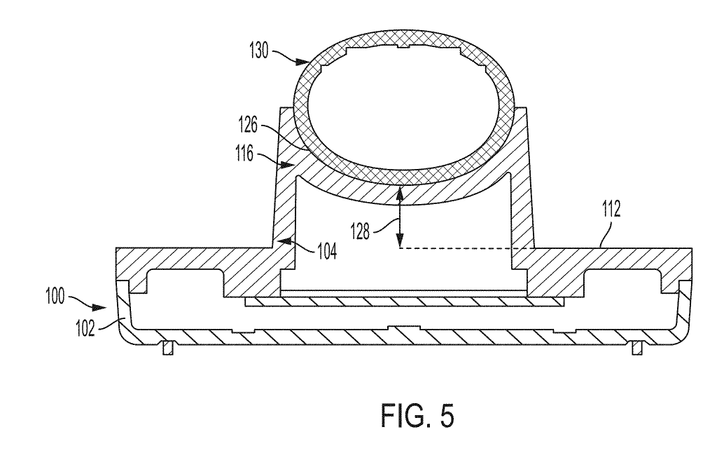

FIG. 5 shows an example cross-section of the charger 100 holding the ultrasound imaging device 130 along the axis B-B of FIG. 3A in accordance with certain embodiments described herein. As can be seen, the second surface 126 of the second cradle member 116 conforms to the ultrasound imaging device 130. Since FIG. 4 and FIG. 5 show cross-sections from a single configuration of the charger 100 and the ultrasound imaging device 130 while the charger 100 holds the ultrasound imaging device 130, it should be appreciated that FIG. 4 and FIG. 5 show that the first surface 122 of the first cradle member 114 and the second surface 126 of the second cradle member 116 may simultaneously conform to the ultrasound imaging device 130.

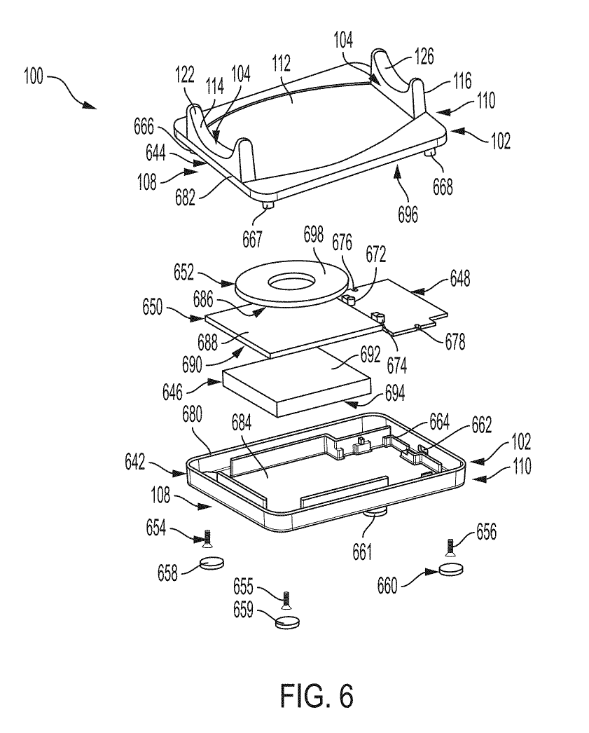

FIG. 6 shows an example exploded view of the charger 100 for wirelessly charging the ultrasound imaging device 130 in accordance with certain embodiments described herein. The charger 100 includes a bottom cover 642, a top cover 644, a compressible element 646, a circuit board 648, a ferrite element 650, and a transmitter coil 652. The bottom cover 642 and the top cover 644, when coupled together, from the base member 102 of FIG. 1. The bottom cover 642 includes screws 654-656 and a fourth screw not visible in FIG. 6, feet 658-661, a slot 662, a protrusion 664 and a second protrusion not visible in FIG. 6, an upper rim 680, and an upper surface 684. The top cover 644 includes screw receptacles 666-668 and a fourth screw receptacle not visible in FIG. 6, the top surface 112, the first cradle member 114, the second cradle member 116, a lower rim 682, and a lower surface 696 (which constitutes the internal top surface of the base member 102). The circuit board 648 includes wire connectors 672 and 674 and notches 676 and 678. The transmitter coil 652 includes a lower surface 686 and an upper surface 698. The ferrite element 650 includes an upper surface 688 and a lower surface 690. The compressible element 646 includes an upper surface 692 and a lower surface 694. In some embodiments, the compressible element 646 includes a rubber material and has a thickness between 0.05-1 inches, (e.g., 0.05, 0.1 0.15, 0.2, 0.25, 0.3, 0.4, 0.5, 0.75, 1 inches, or any suitable thickness.

The bottom cover 642 and the top cover 644, when coupled together such that the upper rim 680 of the bottom cover 642 contacts the lower rim 682 of the top cover 644, form a cavity in the base member 102 that contains the compressible element 646, the circuit board 648, the ferrite element 650, and the transmitter coil 652. While the compressible element 646, the circuit board 648, the ferrite element 650, and the transmitter coil 652 are shown contained within the cavity in the base member 102, other means for coupling the compressible element 646, the circuit board 648, the ferrite element 650, and the transmitter coil 652 to the base member 102 are possible, such as screws, clips, adhesives, or any other suitable coupling means. To couple together the bottom cover 642 and the top cover 644, the screws 654-656 and the fourth screw not visible in FIG. 6 extend through holes (not visible in FIG. 6) in the bottom cover 642 and into the corresponding screw receptacles 666-668 and the fourth screw receptacle not visible in FIG. 6. It should be appreciated that other means for coupling the bottom cover 642 to the top cover 644 are possible, such as with as clips, adhesives, or any other suitable coupling means. The feet 658-661 couple to the screws 654-656 and the fourth screw not visible in FIG. 6. The feet 658-661 are optional. It should be appreciated that other means for coupling the bottom cover 642 to the top cover 644 are possible, such as with as screws, clips, adhesives, or any other suitable coupling means.

The circuit board 648 is secured to the bottom cover 642 by placing the notches 676 and 678 around the corresponding protrusion 664 and the second protrusion not visible in FIG. 6. The slot 662 is configured to allow an external power-supplying cable (such as a USB cable, a microUSB cable, a Lightning cable, a standard power cable, etc.), to extend through the slot 662 in the bottom cover 642 and connect electrically to the circuit board 648 (e.g., by being inserted into a cable receptacle on the circuit board 648). The wire connectors 672 and 674 are configured to electrically connect to wires (not visible in FIG. 6) extending from the transmitter coil 652 and facilitate electrical connection of the circuit board 648 to the transmitter coil 652. The circuit board 648 contains electrical components that transform a voltage supplied by the external power-supplying cable into an alternating current that is supplied to the transmitter coil 652 through the wire connectors 672 and 674.

The transmitter coil 652 is configured to generate a magnetic field based on the alternating current flowing through the transmitter coil 652. The magnetic field extends to a receiver coil in the ultrasound imaging device 130 (not shown in FIG. 6) when the ultrasound imaging device 130 is held by the charger 100 during wireless charging. The magnetic field generated by the transmitter coil 652 wirelessly induces an alternating current in the receiver coil of the ultrasound imaging device 130 that in turn is converted into a direct current by circuit components in the ultrasound imaging device 130. The direct current is used to charge a battery in the ultrasound imaging device 130.

The transmitter coil 652 is coupled to the upper surface of the ferrite element 650. The ferrite element 650 is configured to concentrate the magnetic field on the transmitter coil 652 and the receiver coil in the ultrasound imaging device, and to shield other electrical components in the charger 100 from the generated magnetic field.

The transmitter coil 652, the ferrite element 650, and the compressible element 646 form a stack in which the lower surface 686 of the transmitter coil 652 contacts the upper surface 688 of the ferrite element 650, the lower surface 690 of the ferrite element contacts the upper surface 692 of the compressible element 646, and the lower surface 694 of the compressible element 646 contacts the upper surface 684 of the bottom cover 642. The transmitter coil 652 is disposed adjacent to the lower surface 696 of the top cover 644, and is therefore disposed between the compressible element 646 and the lower surface 696 (where “between” does not preclude other elements being disposed between the compressible element 646 and the lower surface 696).

The compressible element 646 is configured to exert an upward force on the ferrite element 650 which in turn exerts an upward force on the transmitter coil 652, in order to press the transmitter coil 652 (in particular, the upper surface 698 of the ferrite element 650) against the lower surface 696 of the top cover 644. In some embodiments, the height of the stack of the compressible element 646, the ferrite element 650, and the transmitter coil 652 may be larger than the height of the cavity between the lower surface 696 of the top cover 644 and the upper surface 684 of the bottom cover 642 when the bottom cover 642 and the top cover 644 are coupled together. Therefore, when the bottom cover 642 and the top cover 644 are coupled together with the compressible element 646, the ferrite element 650, and the transmitter coil 652 in between, the compressible element 646 may be able to compress downwards in height to the height of the cavity and exert an upward force on the ferrite element 650 which in turn exerts an upward force on the transmitter coil 652, in order to press the transmitter coil 652 against the lower surface 696 of the top cover 644. Pressing the transmitter coil 652 against the lower surface 696 of the top cover 644 may help to ensure that the transmitter coil 652 is positioned as close as possible to the receiver coil of the ultrasound imaging device 130 being held by the charger 100 (in particular, by the first cradle member 114 and the second cradle member 116). Positioning the transmitter coil 652 as close as possible to the receiver coil of the ultrasound imaging device 130 may help to increase energy transfer efficiency between the transmitter coil 652 and the receiver coil of the ultrasound imaging device 130.

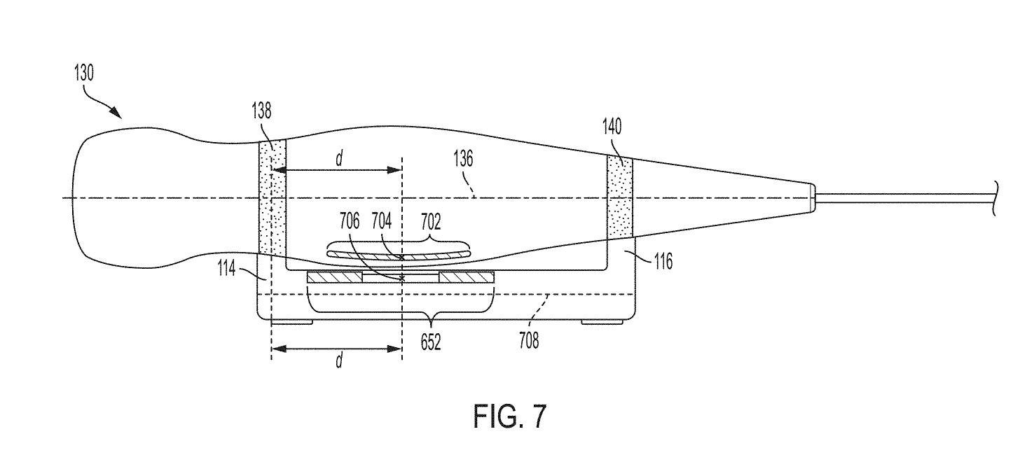

FIG. 7 shows an example cross-section of the charger 100 holding the ultrasound imaging device 130 along the axis C-C of FIG. 3B. For simplicity, certain internal components and structures of the charger 100 and the ultrasound imaging device 130 have been omitted. In particular, FIG. 7 shows the transmitter coil 652 of the charger 100 and a receiver coil 702 of the ultrasound imaging device 130. The transmitter coil 652 has a center portion 706 at substantially the center of the transmitter coil 652. The receiver coil 702 has a center portion 704 at substantially the center of the receiver coil 702. The charger 100 includes a longitudinal axis 708 and the ultrasound imaging device 130 includes the longitudinal axis 136.

The cradle 104 may configured to hold the ultrasound imaging device 130 so as to substantially maintain alignment of the receiver coil 702 of the ultrasound imaging device 130 with the transmitter coil 652 of the charger 100. In particular, as shown in FIG. 7, the distance between the first portion 138 of the ultrasound imaging device 130 and the center portion 704 of the receiver coil 702 along the longitudinal axis 136 of the ultrasound imaging device is d. Furthermore, the distance between the first cradle member 114 and the center portion 706 of the transmitter coil 652 along the longitudinal axis 708 of the charger 100 is also d. Furthermore, as discussed above, various dimensions of the charger 100 may be selected such that there may only be one position of the ultrasound imaging device 130 relative to the charger 100 in which the first surface 122 and the second surface 126 simultaneously conform to the ultrasound imaging device 130 in some embodiments. For example, the only portions of the ultrasound imaging device 130 to which the first surface 122 and the second surface 126 can simultaneously conform may be the first portion 138 and the second portion 140 of the ultrasound imaging device 130, respectively. In such embodiments, when the first surface 122 and the second surface 126 do simultaneously conform to the ultrasound imaging device 130, the center portion 704 of the receiver coil 702 will be positioned a distance d from the first cradle member 114, because the first cradle member 114 will be holding the first portion 138 of the ultrasound imaging device 130, which is positioned a distance d from the center portion 704 of the receiver coil 702, as shown in FIG. 7. Furthermore, because the distance between the first cradle member 114 and the center portion 706 of the transmitter coil 652 is also d, the center portion 704 of the receiver coil 702 will be positioned substantially directly above the center portion 706 of the transmitter coil 652. Accordingly, a user can know that when the ultrasound imaging device 130 is placed in the charger 100 such that the first surface 122 and the second surface 126 do simultaneously conform to the ultrasound imaging device 130, the center portion 704 of the receiver coil 702 will be positioned substantially directly above the center portion 706 of the transmitter coil 652. Ensuring that a user can know when the center portion 704 of the receiver coil 702 is positioned substantially directly above the center portion 706 of the transmitter coil 652 based on whether the first surface 122 and the second surface 126 simultaneously conform to the ultrasound imaging device 130 may be helpful. In particular, positioning the center portion 704 of the receiver coil 702 directly above the center portion 706 of the transmitter coil 652 may help to decrease the distance from the receiver coil 702 to the transmitter coil 652 and thereby increase energy transfer efficiency between the transmitter coil 652 and the receiver coil of the ultrasound imaging device 130. It will be appreciated that the above discussion applies equally if the charger 100 is configured such that the distance between the second cradle member 116 and the center portion 706 of the transmitter coil 652 is the same as the distance between the second portion 140 of the ultrasound imaging device 130 and the center portion 704 of the receiver coil 702.

The cradle 104 may be configured to substantially maintain alignment of the receiver coil 702 of the ultrasound imaging device 130 with the transmitter coil 652 of the charger 100 by being configured to hold the ultrasound imaging device 130 such that the center portion 704 of the receiver coil 702 is positioned substantially directly above the center portion 706 of the transmitter coil 652. The center portion 704 of the receiver coil 702 may be positioned substantially directly above the center portion 706 of the transmitter coil 652 when the distance between the center portion 704 of the receiver coil 702 and the center portion 706 of the transmitter coil 652 within the plane of the longitudinal axis 708 of the ultrasound imaging device 130 is less than a threshold distance. For example, the threshold distance may be 1/64 inch, 1/32 inch, 1/16 inch, ⅛ inch, ¼ inch, ½ inch, or 1 inch, although other threshold distances are possible.

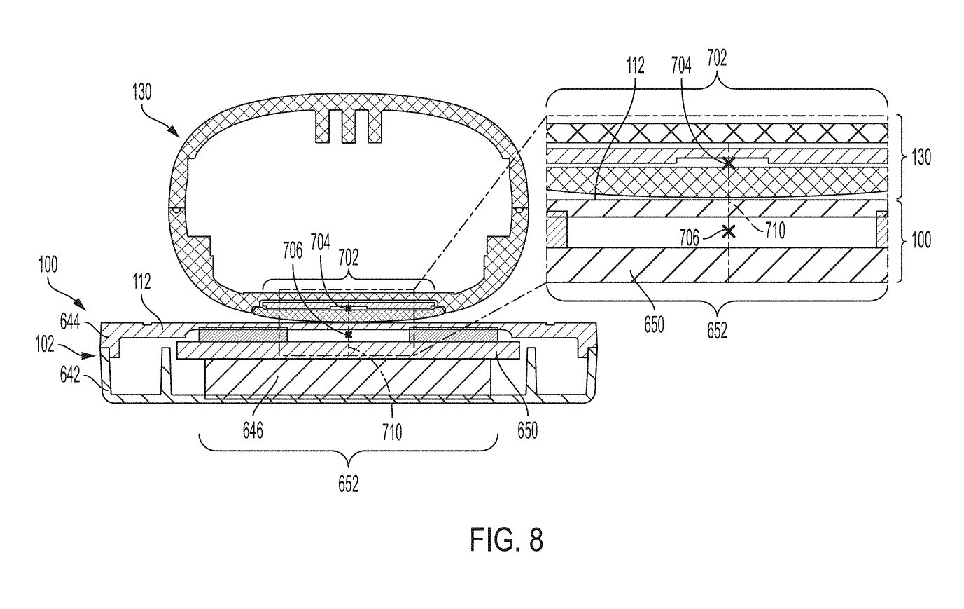

FIG. 8 shows an example cross-section of the charger 100 holding the ultrasound imaging device 130 along the axis D-D of FIG. 3A. In FIG. 8, the charger 100 conforms to the ultrasound imaging device 130 and therefore the center portion 704 of the receiver coil 702 is substantially directly above the center portion 706 of the transmitter coil 652, as indicated by line 710. FIG. 8 also includes a magnification of the charger 100 and the ultrasound imaging device 130 which shows that the ultrasound imaging device 130 does not contact the top surface 112 of the base member 102. In some embodiments, the vertical distance from the center portion 704 of the receiver coil 702 to the center portion 706 of the transmitter coil 652 is between approximately 0.03-0.3 inches (e.g., 0.03, 0.04, 0.05 inches, 0.75 inches, 0.1 inches, 0.15 inches, 0.2 inches, 0.25 inches, 0.3 inches, or any suitable distance).

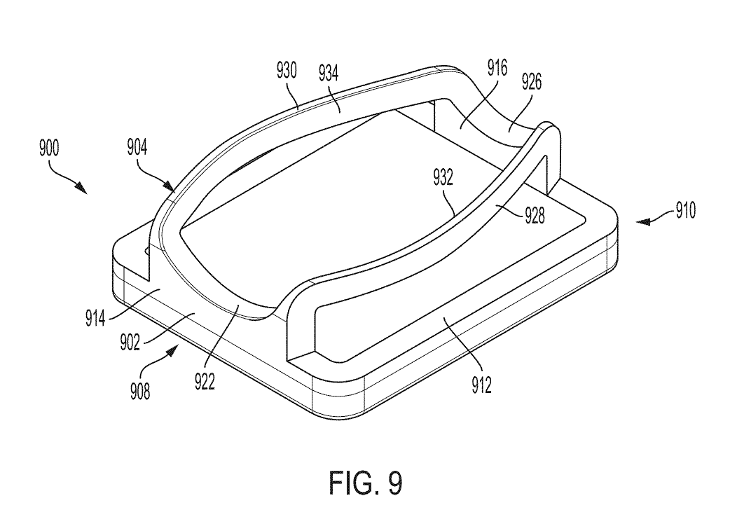

FIG. 9 shows another example charger 900 in accordance with certain embodiments described herein. The charger 900 includes a base member 902 and a cradle 904. The base member 902 includes a first end 908, a second end 910, and a top surface 912. The cradle 904 includes a first cradle member 914 at the first end 908 of the base member 902 and a second cradle member 916 at the second end 910 of the base member 902, with both the first cradle member 914 and the second cradle member 916 extending upwards from the top surface 912 of the base member 902. The first cradle member 914 includes a first surface 922 facing upwards from the top surface 912 of the base member 902 and the second cradle member 916 includes a second surface 926 facing upwards from the top surface 912 of the base member 902. The cradle 904 further includes a first rail 928 extending between the first cradle member 914 and the second cradle member 916, and a second rail 930 extending between the first cradle member 914 and the second cradle member 916. The first rail 928 includes a third surface 932 facing inwards towards the center of the charger 900 and the second rail 930 includes a fourth surface 934 facing inwards towards the center of the charger 900. The first surface 922 and the second surface 926 are configured to hold the ultrasound imaging device 130 such that the ultrasound imaging device 130 may rest on the first surface 922 and the second surface 926. The first rail 928 and the second rail 930 are configured such that when the charger 900 holds the ultrasound imaging device 130, the ultrasound imaging device 130 contacts the third surface 932 and the fourth surface 934, or a portion thereof. The first rail 928 and the second rail 930 are thereby configured to constrain the position and orientation of the ultrasound imaging device 130 relative to the charger 900 such that the center portion 704 of the receiver coil 702 is positioned substantially directly above the center portion 706 of the transmitter coil 652, as discussed above.

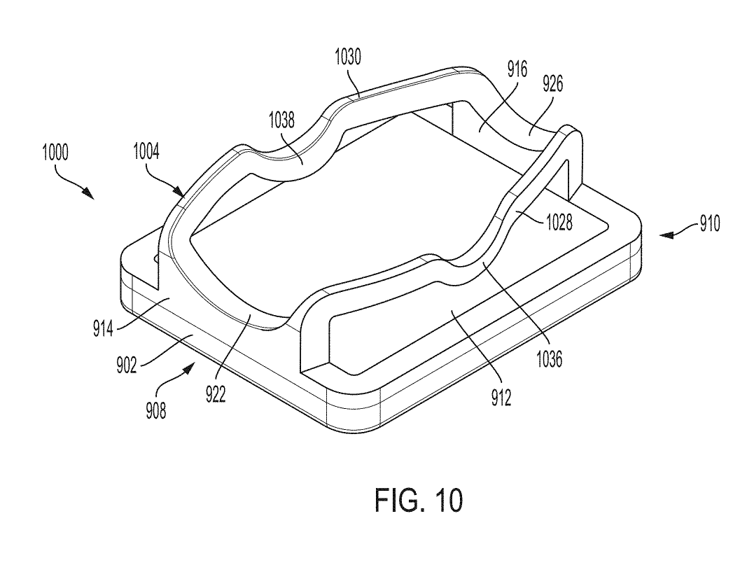

FIG. 10 shows another example charger 1000 in accordance with certain embodiments described herein. The charger 1000 differs from the charger 900 in that the charger 1000 includes a cradle 1004 having a first rail 1028 and a second rail 1030 in which the first rail 1028 includes a first protrusion 1036 and the second rail 1030 includes a second protrusion 1038. The first protrusion 1036 protrudes downwards from the first rail 1028 towards the top surface 912 and the second protrusion 1038 protrudes downwards from the second rail 1030 towards the top surface 912. The first protrusion 1036 and the second protrusion 1038 thereby create spaces such that when the charger 1000 holds the ultrasound imaging device 130, a user may place his/her fingers at the spaces to grip the ultrasound imaging device 130 and remove the ultrasound imaging device 130 from the charger 1000.

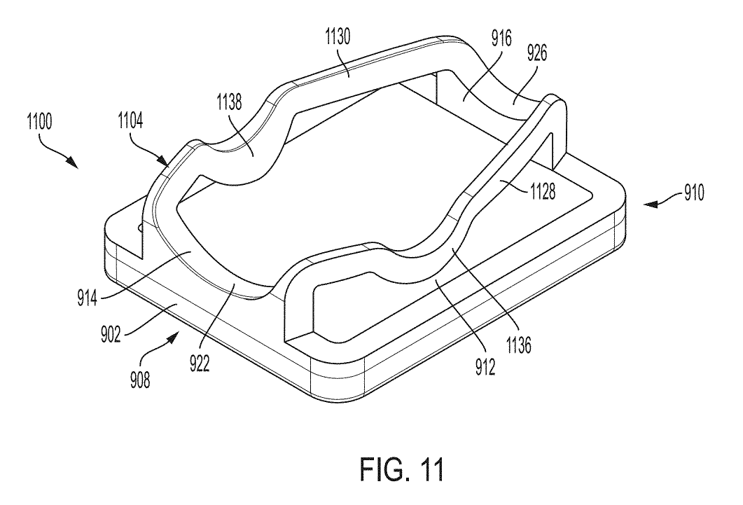

FIG. 11 shows another example charger 1100 in accordance with certain embodiments described herein. The charger 1100 differs from the charger 1000 in that the charger 1100 includes a cradle 1104 having a first rail 1128 and a second rail 1130 that include a first protrusion 1136 and a second protrusion 1138, respectively, that are located at different portions of the first rail 1128 and the second rail 1130 than the first protrusion 1036 and the second protrusion 1038 of the charger 1000. The positioning of the first protrusion 1136 and the second protrusion 1138 may be such that the first protrusion 1136 and the second protrusion 1138 align with specific portions of the ultrasound imaging device 130 (e.g., portions that are wide in diameter) that may facilitate easier gripping and removal of the ultrasound imaging device 130 by a user.

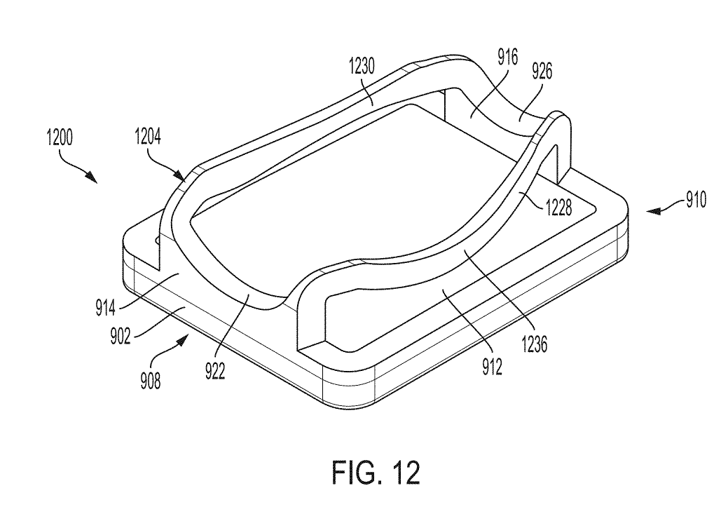

FIG. 12 shows another example charger 1200 in accordance with certain embodiments described herein. The charger 1200 differs from the charger 900 in that the charger 1200 includes a cradle 1204 having a first rail 1228 and a second rail 1230 in which the first rail 1228 curves downwards from the first cradle member 914 and the second cradle 916, towards the top surface 912. The curvature of the first rail 1228, like the protrusions of the chargers 1000 and 1100, may facilitate easier gripping and removal of the ultrasound imaging device 130 by a user.

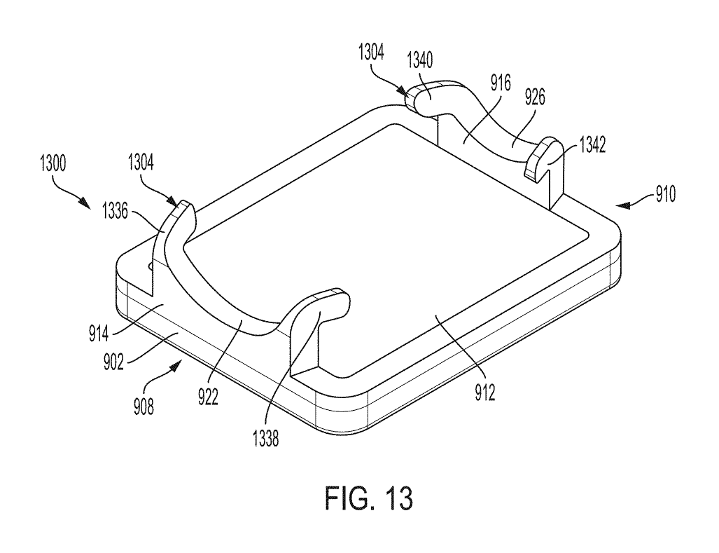

FIG. 13 shows another example charger 1300 in accordance with certain embodiments described herein. The charger 1300 differs from the charger 900 in that the charger 1300 includes a cradle 1304 having a first protrusion 1336 and a second protrusion 1338 extending from the first cradle member 914 and a third protrusion 1340 and a fourth protrusion 1342 extending from the second cradle member 916, instead of the first rail 928 and the second rail 930. The first protrusion 1336 and the second protrusion 1338 extend towards the second end 910 of the base member 902 and the third protrusion 1340 and the fourth protrusion 1342 extend towards the first end 908 of the base member 902. Like the first rail 928 and the second rail 930, the first protrusion 1336, the second protrusion 1338, the third protrusion 1340, and the fourth protrusion 1342 are configured to constrain the position and orientation of the ultrasound imaging device 130 relative to the charger 900 such that the center portion 704 of the receiver coil 702 is positioned substantially directly above the center portion 706 of the transmitter coil 652. In contrast to the first rail 928 and the second rail 930, the first protrusion 1336, the second protrusion 1338, the third protrusion 1340, and the fourth protrusion 1342 do not extend all the way between the first cradle member 914 and the second cradle member 916, and therefore between the first cradle member 914 and the second cradle member 916 there is space for a user to grip and remove the ultrasound imaging device 130 from the charger 1300.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus for wirelessly charging an ultrasound imaging device, comprising:
a base member having a top surface;
a transmitter coil disposed within the base member; and
a cradle extending from the top surface of the base member and configured to hold the ultrasound imaging device so as to substantially maintain alignment of a receiver coil of the ultrasound imaging device with the transmitter coil.

2. The apparatus of claim 1, wherein:
the cradle is configured to hold the ultrasound imaging device such that a center portion of the receiver coil is positioned substantially directly above a center portion of the transmitter coil.

3. The apparatus of claim 2, wherein the cradle further comprises:
a first cradle member having a first surface configured to conform to a first portion of the ultrasound imaging device; and
a second cradle member having a second surface configured to conform to a second portion the ultrasound imaging device;
wherein the first surface of the first cradle member and the second surface of the second cradle member are configured to simultaneously conform to the first portion and the second portion of the ultrasound imaging device, respectively, so as to position the center portion of the receiver coil substantially directly above the center portion of the transmitter coil.

4. The apparatus of claim 3, wherein:
the first surface of the first cradle member is concave and faces a same direction as the top surface of the base member; and
the second surface of the second cradle member is concave and faces the same direction as the top surface of the base member.

5. The apparatus of claim 3, wherein a first radius of curvature of the first surface of the first cradle member is greater than a second radius of curvature of the second surface of the second cradle member.

6. The apparatus of claim 3, wherein a width of the first surface is between approximately 1/16-2 inches.

7. The apparatus of claim 3, wherein a distance between the first cradle member and the second cradle member along a longitudinal axis of the base member is between approximately 2-5 inches.

8-17. (canceled)

18. An apparatus for wirelessly charging an ultrasound imaging device, comprising:
a base member having a top surface;
a transmitter coil disposed within the base member; and
a cradle extending from the top surface of the base member and configured to hold the ultrasound imaging device such that no portion of the ultrasound imaging device contacts the top surface of the base member.

19. The apparatus of claim 18, wherein the cradle comprises a first cradle member having a first surface that is concave and configured to receive a first portion of the ultrasound imaging device.

20. The apparatus of claim 19, wherein:
the first surface comprises a first portion that is substantially closest to the top surface of the base member and is disposed a first vertical distance above the top surface of the base member.

21. The apparatus of claim 19, wherein the cradle further comprises a second cradle member spaced a distance along the longitudinal axis of the base member from the first cradle member and having a second surface that is concave and configured to simultaneously receive a second portion of the ultrasound imaging device when the first surface of the first cradle member receives the first portion of the ultrasound imaging device.

22. The apparatus of claim 21, wherein:
the second surface comprises a second portion that is substantially closest to the top surface of the base member and is disposed a second vertical distance above the top surface of the base member.

23. The apparatus of claim 21, wherein a first radius of curvature of the first surface of the first cradle member is greater than a second radius of curvature of the second surface of the second cradle member.

24. The apparatus of claim 22, wherein the second vertical distance is greater than the first vertical distance.

25. The apparatus of claim 22, wherein the distance between the first cradle member and the second cradle member along the longitudinal axis of the base member is between approximately 2-5 inches.

26. The apparatus of claim 18, wherein a width of the first surface is between approximately 1/16-2 inches.

27-37. (canceled)

38. An apparatus for wirelessly charging an ultrasound imaging device, comprising:
a base member having a top surface;
a transmitter coil disposed within the base member; and
a cradle extending from the top surface of the base member and having a first surface configured to receive the ultrasound imaging device, wherein the first surface comprises a first portion that is substantially closest to the top surface of the base member and is disposed a first vertical distance above the top surface of the base member.

39. The apparatus of claim 38, wherein:
the cradle comprises a first cradle member;
the first cradle member comprises the first surface; and
the first surface is concave and faces a same direction as the top surface of the base member.

40. The apparatus of claim 39, wherein:
the cradle further comprises a second cradle member;
the second cradle member comprises a second surface configured to simultaneously receive the ultrasound imaging device when the first surface of the first cradle member receives the ultrasound imaging device;
the second surface is concave and faces the same direction as the top surface of the base member; and
the second surface comprises a second portion that is substantially closest to the top surface of the base member and is disposed a second vertical distance above the top surface of the base member.

41. The apparatus of claim 40, wherein a first radius of curvature of the first surface of the first cradle member is greater than a second radius of curvature of the second surface of the second cradle member.

42. The apparatus of claim 40, wherein the second vertical distance is greater than the first vertical distance.

43. The apparatus of claim 40, wherein a distance between the first cradle member and the second cradle member is between approximately 2-5 inches.

44. The apparatus of claim 40, wherein a width of the first surface is between approximately 1/16-2 inches.

45-57. (canceled)
